(19) United States
(12) Patent Application Publication
(10) Pub. No.: US 2026/0096915 A1
(43) Pub. Date: Apr. 9, 2026

(54) FALL-PREVENTION FUNCTIONAL LIMITATION DEVICE

(71) Applicants: Gianmarco ROMANELLO, Rome (IT); Andrea ROMANELLO, Rome (IT); Giulio NAPOLITANO, Bracciano (RM) (IT); Daniele ROMANELLO, Rome (IT); VOLKANUS SRL, Rome (IT)

(72) Inventor: Daniele ROMANELLO, Rome (IT)

(73) Assignee: VOLKANUS SRL, Rome (IT)

(21) Appl. No.: 19/111,780

(22) PCT Filed: Sep. 14, 2023
(86) PCT No.: PCT/IB2023/059135

§ 371 Date: Mar. 13, 2025

(30) Foreign Application Priority Data
Sep. 14, 2022 (IT) 102022000018843

(51) Int. Cl. A61F 5/37

(52) U.S. Cl. CPC A61F 5/3784

(57) ABSTRACT

A system for one or more of avoiding and preventing bedridden people from falling. The system includes a bed and a fall-prevention functional limitation device adapted to be wrapped around the bedridden patient. The fall-prevention functional limitation device includes a long smock provided with a cape and two pairs of side bands, an upper pair placed at the lateral ends of the cape and a lower pair placed in the lower part of the smock, respectively. Both pairs of side bands include the respective bands in mirroring position with respect to the longitudinal plane of symmetry of the fall-prevention functional limitation device itself, which is also provided with a hole for the passage of the patient's head.

The object of the present invention is a device adapted to avoid and prevent people falling out of bed who, for pathological reasons or necessity, are bedridden.

The invention thus is usable in all those contexts in which there is a bedridden patient, such as, for example, hospitals, nursing homes, but it is also usable within a domestic context, where a bedridden person is still present.

Means are known for constraining people to bed, providing the use of safety sides to be applied to the edges of the bed so as to form a substantially impassable barrier which prevents the patient from falling out of bed.

However, the sides system is not capable of completely preventing falls because a particularly shaken subject could always climb over the sides and fall out of bed.

Further, although the sides are removable, if necessary, they are a burden for managing the bedridden person because the personnel have to remove them and put them back each time there is a need to provide the patient/subject/person with treatments, medicaments or hygienic treatments.

Optionally, in addition to the above sides, constraint means may be provided when the patient demonstrates particular states of psychomotor agitation or if the patient is dangerous to her/himself or the people assisting her/him.

Often, such constraint means consist of restraints to be applied to ankles and or wrists so as to drastically limit the patient's movements by reducing eliminating the possibility for the patient to climb over the sides and fall out of bed.

However, the known constraint system described above may result in physical or psychological harm and may cause a worsening of the patient's mental state.

It is the task of the invention to overcome the limits of the prior art by providing a device capable of avoiding and preventing patients from accidentally falling without limiting the mobility thereof and therefore, not causing the psychological worsening of the patient.

This has been obtained, according to the present invention, by providing a sort of suitably shaped “smock” which has bands (or strips) which allow constraining the patient subject person to the bed without excessively limiting the movements thereof and keeping the limbs of the bedridden patient'subject person free to move.

For the purposes of the present description, the term “smock” according to the invention means a sheet of sturdy fabric (and not an item of clothing) which covers the patient's whole body at the front. from the head to the feet, and reaches the foot of the bed to be wrapped under the mattress as it were a bed sheet. The length of the sheet on the opposite side is such as to be wrapped behind the subject's shoulders (having inserted the head in a specific opening) to cover the back at least up to under the shoulder blades.

A better understanding of the invention will be achieved by means of the following detailed description and with reference to the accompanying drawings, which show a preferred embodiment of the invention given by way of non-limiting example.

In the drawings:

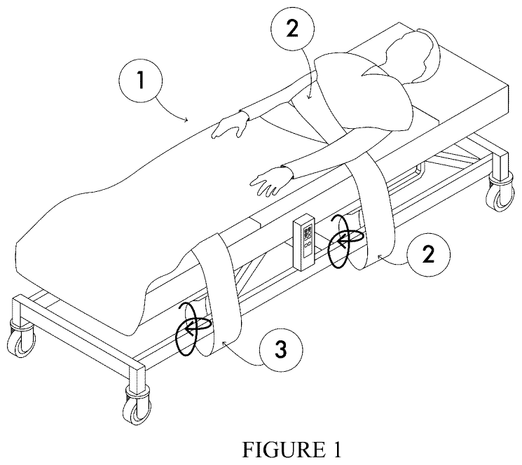

FIG. 1 shows the invention for preventing falls applied to the patient/subject/person.

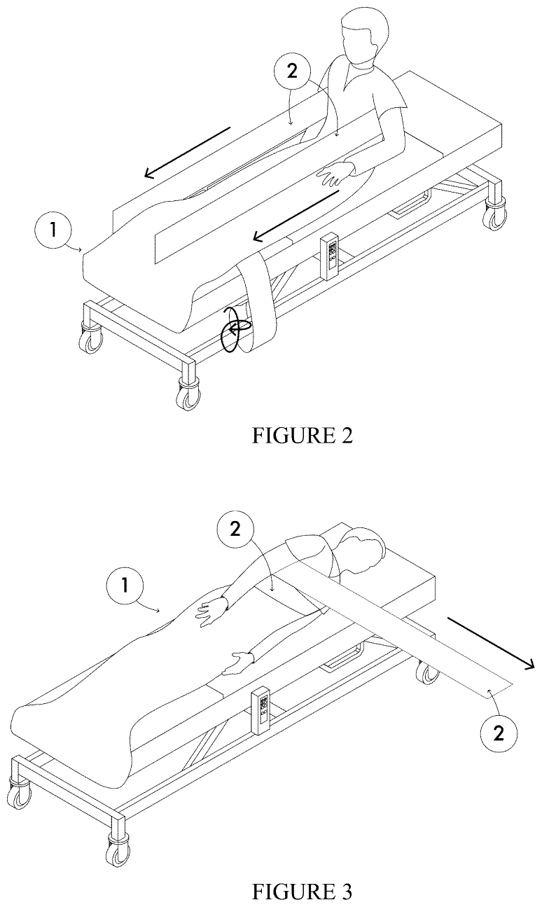

FIG. 2 shows the use of the invention to raise the patient's torso.

FIG. 3 shows the use of the invention to turn the patient onto a side.

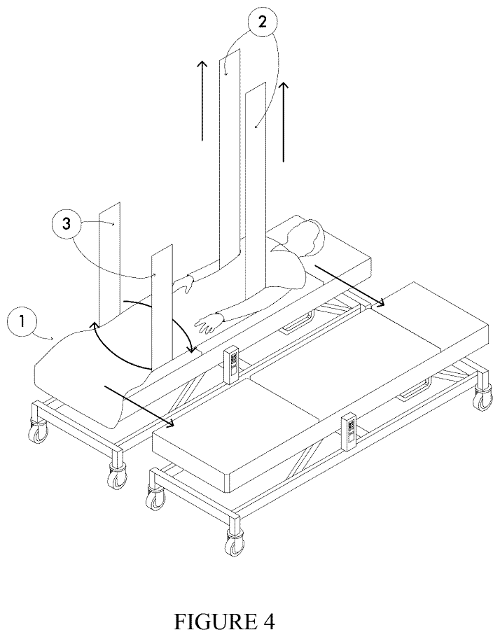

FIG. 4 shows the use of the invention to lift/move the patient.

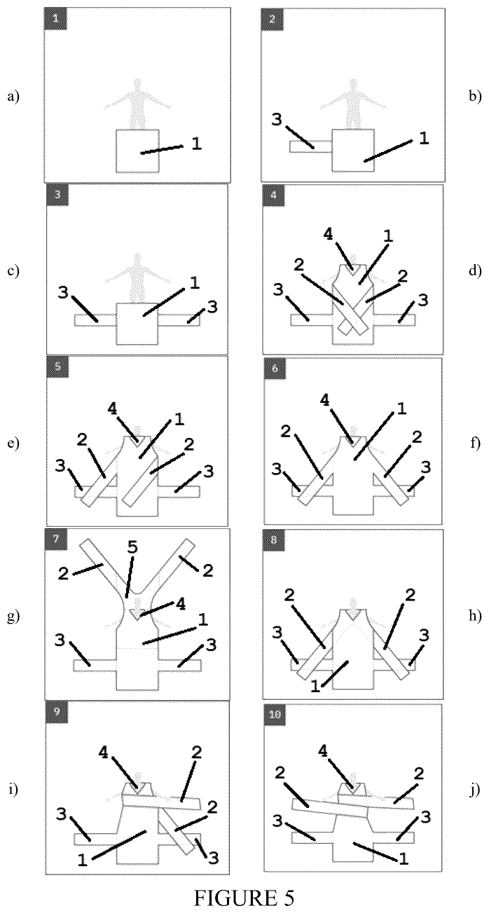

FIGS. 5a, 5b, 5c, 5d, 5e, 5f, 5g, 5h, 5i and 5j show the positioning/application sequence of the invention on the patient's body.

The fall-prevention functional limitation device (1) according to the invention consists of substantially a sheet shaped similarly to a long smock, which is provided with perimeter bands (2, 3) allowing the patient to be wrapped and to constrain her/him to the hospital bed while avoiding and preventing accidental falls, albeit without excessively limiting the patient's movements.

In particular, the smock (1) according to the invention has two pairs of side bands, an upper pair (2) and a lower pair (3), respectively, both pairs having the bands in mirroring position with respect to the longitudinal plane of symmetry of the fall-prevention functional limitation device itself, which corresponds to the plane of symmetry of the person to which it is applied.

Said smock (1) is provided with a hole (4) for the passage of the head of the person patient to which it is applied, and a cape (5) adapted to be turned at the back on the patient's shoulders, at the lateral ends of which the pair of inclined upper bands (2) is fastened (sewn, for example).

The pair of transverse lower bands (3) is instead fastened laterally (sewn, for example) on the lower part of smock (1), at the lower limbs.

Once the smock (1) is wrapped around the patient, thus covering the front part of the body and the rear part at the shoulders by virtue of the presence of cape (5), the pair of upper bands (2) is at the patient's back, while the pair of lower bands (3) is at the lower limbs.

The pair of upper bands (2) therefore may be crossed at the front on the patient's chest and subsequently secured to a side of the bed, i.e., binding the right band to the left side of the bed and the left band to the right side of the bed.

The lower bands (3) may be secured in the same manner.

Practically, the side bands, both upper (2) and lower (3), are directly or indirectly secured to the bed structure by tying the free ends thereof. If, for example, there are safety sides, the ends of said side hands (2, 3) may be fastened simply to such sides.

Alternatively, said ends of the side bands (2, 3) may be provided with means for facilitating the fastening operation to the bed structure or to the sides: for example, said bands could be provided with buckles (not shown) for said fastening.

The fall-prevention functional limitation device (1) according to the invention therefore allows the reduction of falls and the complications of managing the bedridden patient.

According to a peculiar feature of the invention, the fall-prevention functional limitation device (1) has such a configuration that the patient, who is wrapped in the device hereto described, maintains movement autonomy in the bed. but cannot cross the boundary of the bed because this is prevented by the securing of the side bands (2, 3) directly or indirectly to the bed structure.

Thereby, exclusively the movements which could harm the patient are prevented.

In addition to reducing the risk of falling, the present fall-prevention functional limitation device (1) further allows reducing the occurrence of complications, such as, for example, physical or psychological harm, and improves the management itself of the patient by healthcare care personnel since the particular geometry of the upper (2) and lower (3) bands allows a simpler movement during hygienic treatments, medical treatments and during the bed- stretcher and stretcher-bed transfers.

Indeed, the upper (2) and lower (3) bands of device (1) according to the invention may serve as pullers by removing the band/strap opposite to the side from which the patient is to get out of bed, be transferred or placed on a side, by pulling it towards oneself, thus having a kind of “lever effect” by virtue of the band, where it is necessary to place the patient on a side for hygienic treatments or for other maneuvers for providing assistance (FIG. 3).

Further, the patient may take the seated position (FIG. 2) by acting on both upper bands (2).

Finally, the upper (2) and lower (3) bands may serve as an aid for bed- stretcher and stretcher-bed transport; advantageously, this peculiarity is particularly useful in both the nursing and home care assistance of a patient affected by hypomobility, thus reducing the risk of workplace accidents and the physical efforts of personnel during the patient movement and transfer operations (FIG. 4).

A second peculiar feature of the invention consists in the presence of specific notches at the lower vertex of opening (4) for the head, associated with the weaving features. which are configured to allow a quick disengagement of the constraint and an easy and immediate access to the chest to provide possible assistance and emergency treatment.

In a preferred but non-limiting embodiment, device (1) may be made of fabric.

Optionally, said safety device (1) may be made of Tencel/Lyocell.

The outline with opening (4) for the head and the related safety notches made from the lower vertex of the opening itself are obtained from the single piece of fabric.

For reasons of cost savings and use of the fabric, the upper (2) and lower (3) bands or strips are preferably cut out separately and sewn to the main body of device (1).

Finally, it is worth noting that the fall-prevention functional limitation device hereto described is advantageously conceived and obtained to be as manageable, in procuring, using and washing, as a common bed sheet so as to have overlapping management costs. This is made possible by the manufacturing simplicity thereof which does not provide additional components such as laces, strings, zippers or other additions to the basic fabric.

1. A system that one or more of avoids and prevents bedridden people from falling while allowing freedom of movement of limbs of said bedridden people, said system comprising:
a bed; and
a fall-prevention functional limitation device configured to be wrapped around a patient,
wherein said fall-prevention functional limitation device comprises
a long smock comprising a cape and two pairs of side bands,
wherein said two pairs of side bands comprise an upper pair of bands placed at lateral ends of the cape, and a lower pair of bands placed in a lower part of the long smock, respectively;
wherein said two pairs of side bands comprise respective bands in mirroring position with respect to a longitudinal plane of symmetry of the fall-prevention functional limitation device itself; and,
a hole that allows passage of a head of the patient;
wherein said two pairs of side bands are configured to be used to move the patient.

2. The system according to claim 1, wherein the pair of upper bands are configured to be wrapped at a front around a chest of the patient and a length of each upper band of said upper pair of bands is such that a free end of said each upper band is directly or indirectly constrained to a bed structure on an opposite side of the bed itself.

3. The system according to claim 1, wherein the lower pair of bands are configured to be directly or indirectly constrained to a bed structure or wrapped at a back around lower limbs of the patient and a length of each lower band of said lower pair of bands is such that a free end of said each lower band is directly or indirectly constrainable to the bed structure on an opposite side of the bed itself.

4. The system according to claim 1, wherein said two pairs of side bands are directly or indirectly secured to a bed structure via simple binding or via fastening means.

5. The system according to claim 4, wherein said fastening means comprises buckles.

6. The system according to claim 1, wherein said fall-prevention functional limitation device is made of fabric.

7. The system according to claim 1, wherein said fall-prevention functional limitation device is made of lyocell.

8. The system according to claim 1, wherein said long smock further comprises a sheet of sturdy fabric configured to cover a whole body of the patient at a front, from the head of the patient to feet of the patient and up to a foot of the bed, and configured to be wrapped under a mattress; while a length of the sheet on an opposite side is such as to be wrapped behind shoulders of the patient to cover a back of the patient at least up to under shoulder blades of the patient.
